SEQUENCE LISTING (4 sequences)

SEQ ID NO: 1 - protein, 61 aa, Homo sapien
MDPNCSCSPVGSCACAGSCKCKECKCTSCKKSCCSCCPVGCAKCAQGCICKGTSDKCSCCA

SEQ ID NO: 2 - protein, 61 aa, Homo sapien
MDPNCSCAAGDSCTCAGSCKCKECKCTSCKKSCCSCCPVGCAKCAQGCICKGASDKCSCCA

SEQ ID NO: 3 - protein, 68 aa, Homo sapien
MDPETCPCPSGGSCTCADSCKCEGCKCTSCKKSCCSCCPAECEKCAKDCVCKGGEAAEAEAEKCSCCQ

SEQ ID NO: 4 - protein, 62 aa, Homo sapien
MDPRECVCMSGGICMCGDNCKCTTCNCKTCRKSCCPCCPPGCAKCARGCICKGGSDKCSCCP

(19) United States
(12) Patent Application Publication
(10) Pub. No.: US 2009/0318333 A1
(43) Pub. Date: Dec. 24, 2009

(54) Modified Metallothioneins and Methods for Screening and Treatment of Diseases Associated With Oxidative Stress

(75) Inventor: Bert L. Vallee, Boston, MA (US)

Correspondence Address: CLARK & ELBING LLP, 101 FEDERAL STREET, BOSTON, MA, 02110, US

(73) Assignee: President and Fellows of Harvard College, Cambridge, MA (US)

(21) Appl. No.: 12/225,780

(22) PCT Filed: Mar. 29, 2007
(86) PCT No.: PCT/US07/07581

§ 371 Date: May 18, 2009

Related U.S. Application Data
(60) Provisional application No. 60/787,400, filed on Mar. 30, 2006.
(60) Provisional application No. 60/839,582, filed on Aug. 23, 2006.

(51) Int. Cl. A61K 38/17; C07K 14/825; G01N 33/20; G01N 33/53; A61P 25/28

(52) U.S. Cl. 514/6; 530/400; 436/73; 435/7.1

(57) ABSTRACT

The present invention is based on the therapeutic potential of a reduced form of thionein. Accordingly, the invention features modified metallothionein or thionein proteins, for example, where at least one sulfur atom is substituted with selenium (e.g., a cysteine substituted with selenocysteine), and fragments thereof. The invention also features methods for screening for candidate compounds that (i) decrease binding of metal (e.g., zinc) to metallothionein or thionein and (ii) do not change the oxidation state of metallothionein, thionein, or another protein. Also featured are methods for generating modified thionein proteins with reduced metal affinity and methods for treating patients with a disease associated with oxidative stress.

### BACKGROUND OF THE INVENTION

The invention relates to methods and treatments for diseases associated with oxidative stress and modified metallothionein or thionein proteins which can be useful in such methods.

The family of metallothionein proteins were initially identified as being metal binding proteins, including zinc. However, while zinc is not a redox active metal, by virtue of their unique grouping of cysteine residues capable of metal binding (e.g., zinc), metallothionein and thionein may participate in redox reactions. Accordingly, prior to the present invention, oxidation of metallothionein and release of bound metal have been closely linked.

Diseases associated with oxidative stress include Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis. As many of these diseases have no cure, new methods for identifying treatments for such diseases are needed.

### SUMMARY OF THE INVENTION

The present invention is based on the therapeutic potential of a reduced form of thionein.

The invention accordingly features metallothionein, thionein, or a fragment thereof where one or more sulfur atoms has been substituted with selenium. For example, 1, 2, 3, 4, 5, 6, 7, 8, 9, 10, 11, 12, 13, 14, 15, 16, 17, 18, 19, 20, 21, or more cysteines may be substituted with selenocysteines (e.g., at any or all cysteines or methionines, as described herein). In one embodiment, the invention features a fragment (e.g., an α-domain or a β-domain of metallothionein) of metallothionein capable of binding metal (e.g., selected from the group consisting of main group metals, transition metals, lanthanides, and actinides or selected from the group consisting of zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, or lanthanum) where at least one (e.g., all) sulfur atoms are substituted with selenium (e.g., any substitution described herein). The sulfur atoms in any of the polypeptides described herein may be in a cysteine.

The invention also features a method for identifying a candidate compound for treatment of a disease associated with oxidative stress (e.g., Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis). The method includes the steps of (a) contacting a compound (e.g., a compound selected from a chemical library) with metallothionein or thionein and a second polypeptide including an amino acid capable of being oxidized and (b) measuring the amount of metal (e.g., a metal selected from the group consisting of main group metals, transition metals, lanthanides, and actinides or selected from the group consisting of zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, and lanthanum) released from the metallothionein or thionein and the formation of an oxidized amino acid (e.g., methionine sulfoxide) on the second polypeptide (e.g., metallothionein or thionein) in the presence of the compound, where a compound that (i) increases the release of metal from metallothionein or thionein and (ii) does not substantially increase the amount of the oxidized amino acid in the second polypeptide as compared to in the absence of the compound indicates that the compound is a candidate compound for treatment of a disease associated with oxidative stress.

The invention features another method for identifying a candidate compound for treatment of a disease associated with oxidative stress (e.g., Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis). The method includes the steps of (a) contacting a cell or cell extract with a compound (e.g., a compound selected from a chemical library) and (b) measuring the amount metallothionein or thionein in and the oxidation state (e.g., the presence of oxidized amino acids such as methionine sulfoxide) of the cell or cell extract where a compound that (i) increases the amount of thionein (e.g., metal free thionein) or decreases the amount of metallothionein and (ii) does not substantially increase the oxidation state of the cell or cell extract as compared to a cell or cell extract not contacted with the compound indicates that the compound is a candidate compound for the treatment of a disease associated with oxidative stress.

The invention also features a third method for identifying a candidate compound for treatment of a disease associated with oxidative stress (e.g., Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis). The method includes the steps of (a) contacting a compound (e.g., a compound selected from a chemical library) with a cell or cell extract including a polynucleotide encoding thionein, and (b) measuring expression of thionein in the cell or cell extract, where an increase in expression in the presence as compared to in the absence of the compound indicates that the compound is a candidate compound for the treatment of a disease associated with oxidative stress.

In another embodiment, the invention features a method for identifying a thionein variant with a reduced affinity for a metal. The method includes (a) introducing a point mutation, insertion, or deletion into thionein or chemically altering thionein, thereby creating a modified thionein; and (b) determining the affinity of the metal (e.g., selected from the group consisting of main group metals, transition metals, lanthanides, and actinides or selected from the group consisting of zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, or lanthanum) to the modified thionein, where a decreased affinity for the metal indicates that the modified thionein is a thionein variant with reduced affinity for a metal. The determining step may further include measuring the reducing activity of the modified thionein, where no substantial decrease in the reducing activity of the modified thionein indicates that the modified thionein is a redox-active thionein variant with a reduced affinity for metal.

The invention also features methods for treating a disease associated with oxidative stress (e.g., Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis). In one embodiment, the method includes administering a thionein variant identified using the method the previous embodiment to a patient in need thereof. In another embodiment, the method includes administering a chelating agent to the patient, where the patient has a disease selected from Creutzfeldt-Jakob disease, respiratory distress syndrome, dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, or ulcerative colitis.

Any of the methods of the invention may employ any metallothionein variant, fragment, or derivative (e.g., those described herein). In certain embodiments, the MT/T variant has a sulfur atom substituted with a selenium atom, for example, a point mutation comprising a substitution of one or more (e.g., all) cysteines (e.g., those described herein) with selenocysteine.

In any of the compositions or methods of the invention, the MT or T employed may have substitutions of one or more non-cysteine residues with different amino acids (e.g., naturally occurring or non-naturally occurring amino acids). Further, the MT or T employed may have substitutions of one or more sulfur atoms with selenium (e.g., cysteine residues with selenocysteine). MT/T variants useful in the methods and compositions of the invention include one or more repetitions of the primary sequence of the α or β domain of metallothionein (e.g., separated by a spacer sequence of one or more amino acids). Further MT/T variants include any combination of α (e.g., 1, 2, 3, 4, 5, 8, 10, 12, or more) or β (e.g., 1, 2, 3, 4, 5, 8, 10, 12, or more) domains linked in any order, optionally with one or more spacers between the domains. The domains may further contain substitutions of any non-cysteine amino acid or substitution a sulfur atom with selenium (e.g., cysteine with selenocysteine).

By “metallothionein” is meant a protein having at least 50%, 60%, 70%, 80%, 90%, 95%, 98%, 99%, or even 100% identity to any of SEQ ID NOS:1-4, or homologs thereof, and having seven metal atoms bound to the protein.

By “thionein” is meant a protein having at least 50%, 60%, 70%, 80%, 90%, 95%, 98%, 99%, or even 100% identity to any of SEQ ID NOS:1-4, or homologs thereof, and having six or fewer metal atoms bound. By “metal free thionein” is meant thionein having zero metal atoms bound.

By a compound that “increases the release of metal from metallothionein” is meant a compound that increases the amount thionein (e.g., metal free thionein) by at least 5%, 10%, 25%, 50%, 100%, 200%, 500%, 1000% as compared to in the absence of the compound. Alternatively, a compound that “increases the release of metal from metallothionein” may increase the binding constant (i.e., decrease the affinity) of MT/T for zinc by a factor of at least 2, 5, 10, 50, 100, 1000, 10⁴, 10⁵, 10⁶, 10⁷, 10⁸, 10⁹, or 10¹⁰.

By a compound that “does not substantially increase the amount of said oxidized amino acid in said second polypeptide” or means a compound that increases the amount of the oxidized amino acids by less than 1%, 2%, 5%, 10%, 25%, 50%, 100%, or 500% as compared to in the absence of the compound. In some embodiments, the compound does not alter the amount oxidized amino acids or may further decrease the amount of oxidized amino acids.

By a compound which “does not substantially increase the oxidative state” of a cell or cell lysate means that the compound does not increase the redox potential of the cell or cell lysate by more than 0.01, 0.05, 0.10, 0.20, 0.4, 0.5, 0.75, 1, 2, 5, 10, 25, 50, 100, 200, 500, 1000, 1500, 2000, 5000, 10,000, or 20,000 mV.

Other features and advantages of the invention will be apparent from the following Detailed Description, the drawings, and the claims.

### BRIEF DESCRIPTION OF THE DRAWINGS

FIG. 1 is a set of sequences including human metallothionein 1, 2, 3, and 4 sequences (SEQ ID NOS:1-4).

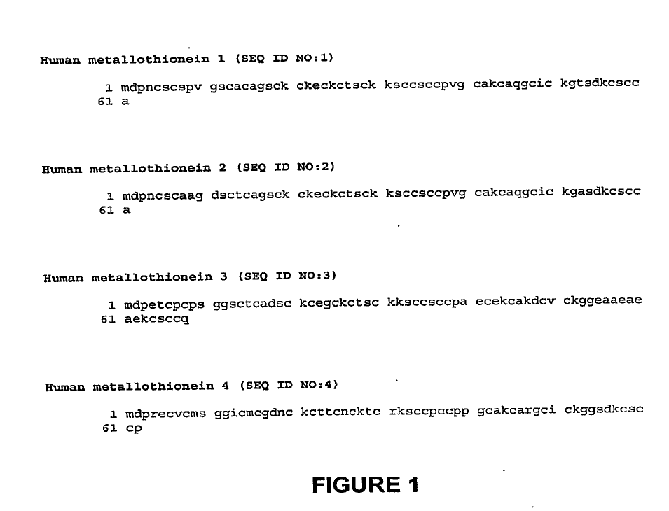

FIGS. 2A-2C are schematic diagrams of zinc clusters in the α-domain of metallothionein (FIG. 2A), in the β-domain of metallothionein (FIG. 2B), and in the GAL4 protein (FIG. 2C).

### DETAILED DESCRIPTION

Of the three metallothionein/thionein species (i.e., metallothionein, oxidized thionein, and reduced thionein), we now believe reduced thionein is a therapeutically important species in diseases associated with oxidative stress. Accordingly, the invention features modified metallothionein and thionein polypeptides, methods to increase levels of reduced thionein, methods for generating thionein variants that disfavor metal binding and, further, favor the reduced state, and methods for treatment of disease associated with oxidative stress which decrease metal binding to thionein as well as increase the amount of reduced thionein available in a subject. The screening methods of invention can identify compounds useful in treatment of diseases associated with oxidative stress (e.g., those described herein).

### Metallothionein and Thionein

Thionein is a 60+ amino acid protein with approximately 20 cysteine amino acids. It contains neither aromatic nor histidine residues. Metallothionein was discovered in 1957 (Margoshes and Vallee, J. Am. Chem. Soc. 79:4813-4814, 1957). Two highly similar forms MT-1 and MT-2 were identified; more recently, a third form MT-3 was identified in brains of Alzheimer's patients (Uchida et al., Neuron 7:337-347, 1991) as a growth inhibitory factor. A fourth variant, MT-4, was also found to be expressed exclusively in stratified squamous epithelia (Quaife et al., Biochemistry 33:7250-9, 1994). Genes coding for additional (as many as seventeen total) MT isoforms have also been identified.

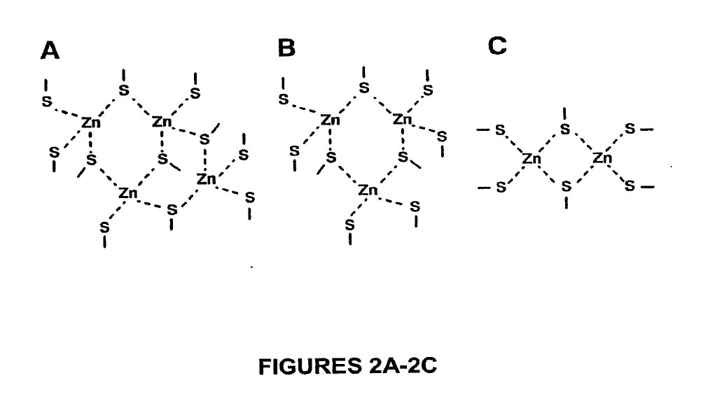

Thionein has two domains (β and α). The N-terminal β domain contains nine cysteines, which can bind three metal atoms (e.g., zinc), and the C-terminal a domain contains 11 cysteines which can bind four metal atoms (e.g., zinc), thereby forming metallothionein (Maret et al., Proc. Natl. Acad. Sci. USA 94:2233-2237, 1997). While zinc enzymes such as GAL4 bind metal in two-zinc clusters (FIG. 2C), metallothionein binds zinc in an unusual manner, through a three-zinc cluster (β domain; FIG. 2B), and a four-zinc cluster (a domain; FIG. 2A). These unusual structures, which possess a high kinetic lability but are thermodynamically stable, are likely related to MT/T cellular function in zinc regulation. In certain embodiments of the invention, the clusters, either as part of a domain, or in smaller portions of either domain comprising a sufficient number of amino acids (e.g., cysteines) to bind metal, may be evaluated for their ability to uptake or release metal (e.g., as described herein) or the equilibria governing their behavior can be evaluated. Metals used in such assays may include any described herein. Binding may be assayed using isotopically labeled transition metals or group IIb metals. Such experiments may be performed with any of MT-1, MT-2, MT-3, MT-4, or any other MT variant, derivative, or fragment (e.g., those described herein). Additional MT variants may be identified and their regulation, expression, or localization may be characterized using any methods known in the art.

### Regulation of Cellular Zinc

One function ascribed to metallothionein and thionein is regulation of cellular zinc levels (Jacob et al., Proc. Natl. Acad. Sci. USA 95:3489-3494, 1998). Zinc is a critical cofactor in numerous enzymes. While zinc binding to MT/T is strong (binding constant of 3.2×10⁻¹³ M at pH 7.4), it has been shown that MT/T can donate zinc to zinc enzymes such as Escherichia coli alkaline phosphatase and bovine carboxypeptidase A.

In addition to being required for enzymatic activity, zinc inhibits activity of some enzymes, including caspase-3, fructose 1,6-diphosphatase, glyceraldehyde 3-phosphate dehydrogenase, aldehyde dehydrogenase, tyrosine phosphatase, and yeast enolase (Maret et al., Proc. Natl. Acad. Sci. USA. 96:1936-1940, 1999). Enzymes inactivated by zinc exhibit restored activity upon addition of metal-free thionein.

### Cellular Oxidation

Previous work (Maret and Vallee, Proc. Natl. Acad. Sci. USA 95:3478-3482, 1998) has shown that, while zinc itself is redox inert, metallothionein and thionein are redox active. Oxidizing agents have been shown to reduce the cysteines in MT/T thereby, causing a concomitant release of metal from the protein. Thus, MT/T are likely involved in maintaining the oxidative state in cells.

### Nucleotide Triphosphate Binding

Nucleotide triphosphates, including ATP, GTP, and ATP analogs such as adenosine 5′[γ-thio]triphosphate and AMP-PNP bind to MT/T, and cause a release of metal from MT/T (Jiang et al., Proc. Natl. Acad. Sci. USA 95:9146-9149, 1998). This binding is mediated through eight conserved lysine residues found in mammalian metallothionein and thionein.

### Identification of Unbound Thionein in Cells

While metallothionein is generally observed in its metal bound form, thionein has been detected in and isolated from biological material (Maret et al., Proc. Natl. Acad. Sci. USA. 96:1936-1940, 1999) and acts as an endogenous chelating agent.

### Additional MT/T Applications

Metallothionein or thionein can be isolated, purified, or fractionated from any organism producing thionein naturally or an organism modified to produce thionein. In certain embodiments, human, bovine, or equine thionein can be isolated, purified, or fractionated. Metallothioneins can also be evaluated for their immunological properties, or their reactive properties (e.g., with any metal described herein, with any nucleotide, nucleoside, or any other compound or polypeptide). In certain embodiments, interactions of MT/T or any variant described herein with AMP, ADP, ATP, GSH, GSSG, or any combination thereof are studied.

The metal loading state of MT/T may also be analyzed, for example, using the methods described in Richarz A N. 2002. Speziationsanalyse von proteingebundenen Elementen in Cytosolen als biologische Marker für Lebensprozesse unter besonderer Berücksichtigung der Metallothioneine im Gehirn (dissertation), Technical University of Berlin of Mathematics and Natural Science: Berlin (DE).

Further, detection of MT/T fractions in different cellular environments can be achieved by separating cellular organelles, for example, based on density centrifugation. In particular, the lysosomes, peroxisomes, mitochondria, endoplasmic recticulum, Golgi apparati, ribosomes, nuclei, or any other subcellular particle may be analyzed in the methods of the invention. In other embodiments, such subcellular particles may be analyzed from heart, liver, brain, kidney, or any other organ. The interactions of MT/T (e.g., any variant such as those described herein) with AMP, ADP, ATP, GSH, GSSG, or any combination thereof may be analyzed. Such methods may be employed using selenocysteine-substituted MT/T, any seleno-derivatives of MT/T, or any variant or fragment of MT/T described herein.

### Selenium-Substituted MT/T

In one aspect, the invention features metallothionein or thionein where one or more sulfur atoms has been substituted with selenium. The substituted metallothionein or thionein may be bound to 0, 1, 2, 3, 4, 5, 6, 7 or more metal atoms. In certain embodiments, cysteines are substituted with selenocysteines (e.g., at any or all of the cysteines as described herein). Selenocysteine has a known physiological distribution, is non-toxic, and is well tolerated. Thus, proteins containing selenocysteine can be useful as therapeutics (e.g., in treating a disease associated with oxidative stress such as those described herein).

Such proteins can be produced by any method known in the art. For example, peptide synthesis can be used to introduce selenocysteine into a protein sequence, e.g., as described in Oikawa et al., Proc. Natl. Acad. Sci. USA 88:3057-3059, 1991. In this example, the cysteine residues in Neurospora crassa copper metallothionein were substituted with selenocysteine. In other embodiments, a cysteine can be substituted with selenocysteine using a semi-synthetic method, e.g., as described in Hondal et al., J. Am. Chem. Soc. 123:5140-5141, 2001 or as described in Dawson and Kent, Annu. Rev. Biochem. 69:923-960, 2000. Other approaches includes modifying the tRNA in an organism to substitute one amino acid for another, e.g., as described in Wang and Schutz, Chem. Commun. 1-11, 2002 and Wang et al., Science 292:498-500, 2001. Any of these approaches, or any other approach known in the art may be used to generate selenocysteine derivatives of MT/T.

### Other MT/T Variants

The invention also features MT or T variants with substitutions of one or more non-cysteine residues with different amino acids (e.g., naturally occurring or non-naturally occurring amino acids). Further, the invention also features MT or T with substitutions of one or more sulfur atoms with selenium (e.g., cysteine residues with selenocysteine). MT/T variants include one or more repetitions of the primary sequence of the α or β domain of metallothionein (e.g., separated by a spacer sequence of one or more amino acids). MT/T variants may include any combination of α (e.g., 1, 2, 3, 4, 5, 8, 10, 12, or more) or β (e.g., 1, 2, 3, 4, 5, 8, 10, 12, or more) domains linked in any order, optionally with one or more spacers between the domains. The domains may further contain substitutions of any non-cysteine amino acid or substitution of cysteine with a selenium containing residue such as selenocysteine. Domains that include changes encompassed by the present invention are described in WO 00/50448, from page 12, line 4 through page 14, line 5, which is hereby incorporated by reference. Changes described in WO 00/50448 likewise may be incorporated into the full length metallothionein protein, any fragment thereof, or any other variant described herein.

Other variants include fragments such as any metallothionein fragment capable of binding a metal atom, for example, portions of the β domain or α domain where the fragment is missing 1-25 amino acids from the C-terminus of the domain, from the N-terminus of the domain, or a mixture thereof. Deletion mutants capable of binding metal can be identified using molecular biological techniques known in the art, and metal binding may be assessed using any method (e.g., as described herein).

### Screening Methods to Identify Candidate Therapeutic Compounds

Based on the identification of metallothionein and thionein as a zinc binding and regulating protein and its role in cellular oxidation in conjunction with MT/T zinc binding, we now seek to separate the zinc binding activity of MT/T from oxidation of MT/T. Accordingly, the invention features screening methods for the identification of compounds that (i) decrease binding of a metal to MT/T and (ii) do not substantially increase the oxidation of MT/T or a second polypeptide. Compounds identified by the methods of the invention can increase availability of thionein available for reduction of potentially harmful oxidative species such as metallothionein or thionein containing methionine sulfoxide residues. Specific examples of diseases where oxidized amino acids play a role in disease progression include Parkinson's disease, where oxidized α-synuclein containing methionine sulfoxide has been identified (Glaser et al., Biochim. Biophys. Acta. 1703:157-69, 2005) and Alzheimer's disease, where oxidized β-amyloid protein containing methionine sulfoxide residues has been identified (Schoneich, Biochim. Biophys. Acta. 1703:111-9, 2005). Other examples of diseases associated with oxidative stress are described herein. Thus, compounds identified by the screening methods of the invention can be useful in the treatment of a disease associated with oxidative stress.

Screening assays to identify compounds that decrease metal binding to MT/T and do not substantially increase the oxidation of MT/T or a second polypeptide can be carried out by standard methods. The screening methods can involve high-throughput techniques. In addition, these screening techniques can be carried out in cultured cells or in organisms such as worms, flies, or yeast.

### Metallothionein

Screening methods of the invention can include the use of any MT/T protein such as proteins homologous to human MT proteins (e.g., MT proteins from mouse, rat, or rabbit). Any form of MT/T (e.g., MT-3 and those described herein) can be used in the methods of the invention. In particular embodiments, the metallothionein or thionein employed in the screening methods of the invention may comprise a selenium in place of one or more sulfur atoms (e.g., selenocysteine in place of one or more cysteine amino acids) found in the wild-type protein. In other embodiments, the screening methods using any variant of metallothionein or thionein described herein. Any concentration of MT/T may be employed in the screening methods that allows for detection of metal release. Any metal, including those selected from the group consisting of main group metals, transition metals, lanthanides, and actinides and those selected from the group consisting of zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, and lanthanum, capable of being bound by thionein can be used to form metallothionein. In some embodiments, metal-binding (e.g., zinc binding) fragments of MT/T are used (e.g., any described herein a fragment comprising the β domain or a domain) in the screening methods. Any MT/T variant, derivative, fragment described herein may also be used in the methods of the invention.

### Detection of Decreased Metal Binding to Metallothionein

The screening methods of the invention include a step determining the release of metal from MT/T or any fragment or variant thereof described herein. Any method known in the art for determining metal release can be used.

In one embodiment, the method described by Maret and Vallee (Proc. Natl. Acad. Sci. USA 95:3478-3482, 1998) is employed. Briefly, a zinc-complexing dye such as 4-(2-pyridylazo)resorcinol (PAR) or 2-carboxy-2′-hydroxy-5′-sulfoformazylbenzene (zincon) can be used to measure release of zinc from MT/T as the spectral properties of these dyes are altered upon zinc binding. In one particular example, a buffered solution containing 100 μM of PAR or zincon is incubated with 1.3 μM zinc-MT. A test compound is added to the solution; changes in absorbance at 500 nm as for PAR or 620 nm for zincon can be measured using a spectrophotometer and compared to the absorbance in the absence of the test compound, where an increase in absorbance indicates a release of zinc from MT/T. If necessary, the absorbance generated by the test compound can be corrected for in the absorbance measurement. Additional molecules useful in detecting free cellular zinc include Zinpyr-1 analogs which are described, for example, in Goldsmith and Lippard, Inorg. Chem. 45:555-561, 2006 and Woodroofe et al., Inorg. Chem. 44:3112-3120, 2005.

In another embodiment, copper-MT is employed in the screening methods of the invention. Here, copper-MT is contacted with a test compound, and release of copper is monitored using 4-(1,4,7,10-tetrathia-13-aza-cyclopentadec-13-yl)-benzene (CTAP-1). CTAP-1 exhibits increased emission at 480 nm upon excitation at 365 nm in the presence of copper as compared to in the absence of copper (Yang et al., Proc. Natl. Acad. Sci. USA. 102:11179-11184, 2005) and is further suitable for use in screening methods employing cells or cell extracts.

Changes in the relative amounts of metallothionein and thionein, and number of metal atoms bound to thionein may also be analyzed, for example, using the methods described in Richarz A N. 2002. Speziationsanalyse von proteingebundenen Elementen in Cytosolen als biologische Marker für Lebensprozesse unter besonderer Berücksichtigung der Metallothioneine im Gehirn (dissertation), Technical University of Berlin of Mathematics and Natural Science: Berlin (DE). Decreases in the metal loading state of metallothionein or thionein or an increase in the amount of metal free thionein in a biological system upon contact of a test compound can indicate that the compound decreases the ability of MT or T to bind metal and can be detected using the described methods. Further, detection of MT/T fractions in different cellular environments can be achieved by separating cellular organelles, for example, based on density centrifugation. In particular, the lysosomes, peroxisomes, mitochondria, endoplasmic recticulum, Golgi apparati, ribosomes, nuclei, or any other subcellular particle may be analyzed in the methods of the invention. In other embodiments, such subcellular particles may be analyzed from heart, liver, brain, kidney, or any other organ. The interactions of MT/T (e.g., any variant such as those described herein) with AMP, ADP, ATP, GSH, GSSG, or any combination thereof may be analyzed. Such methods may be employed using selenocysteine-substituted MT/T, or any other seleno-derivatives of MT/T.

### Detection of Amino Acid Oxidation and Cellular Oxidation State

The screening methods of the invention also include a step of measuring the oxidation of an amino acid of a second polypeptide (e.g., MT, T, or any fragment or variant described herein), or determining whether the test compound substantially increases the oxidation state of a cell (e.g., formation of oxidized amino acids or reactive oxygen species).

For detection of oxidized amino acids, any method known in the art can be employed in the screening methods of the invention. Exemplary detection methods are disclosed in Shacter, Drug Metab. Rev. 32:307-26, 2000. Specific methods for amino acid detection will depend on the particular type of oxidized amino acid being detected.

Oxidation of methionine can result in the formation of methionine sulfoxide, and, in one embodiment, such residues are detected. Detection of methionine sulfoxide is especially useful as virtually all proteins, including thionein and metallothionein, possess an N-terminal methionine residue. Methionine sulfoxide may be detected by the method described in Sochaski et al. (Anal. Chem. 73:4662-7, 2001). Here, samples are hydrolyzed with methanesulfonic acid. The hydrolyzed sample is then separated on a cation exchange column and amino acids are derivatized as their trimethylsilyl esters. The presence of methionine sulfoxide in the sample is then detected by selected ion monitoring-gas chromatography/mass spectrometry, as is known in the art. Compounds that decrease metal (e.g., zinc) binding of MT/T, but do not increase the formation of methionine sulfoxide (e.g., at the N-terminal methionine in MT/T) are considered useful in the invention.

Detection of changes in redox potential in methods of the invention employing cells or a cell extract may also be performed using any method known in the art. The presence of reactive oxygen species, for example, can also be assayed for using commercially available kits, for example, the Image-iT™ LIVE Green Reactive Oxygen Species Detection Kit (Invitrogen). Additional methods for measuring redox potentials in situ are described in Hanson et al., J. Biol. Chem. 279:13044-13053, 2004. Here, green fluorescent protein (GFP) is modified to contain cysteines. The formation of disulfide bonds in the modified GFP results in changes in the fluorescence of the protein in response to changes in redox potential; these changes can be used to monitor changes in redox potential in a cellular environment.

Compounds which reduce metal (e.g., zinc) binding of metallothionein or thionein but do not substantially reduce the ability of the metallothionein or thionein to participate in redox chemistry are considered useful in the invention.

### Screening for Increased Thionein Expression

Any number of methods are available for carrying out screening assays to identify compounds that increase thionein (e.g., MT-3) expression. According to one approach, candidate compounds are added at varying concentrations to the culture medium of cells expressing a polynucleotide coding for metallothionein. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. The level of gene expression in the presence of the candidate compound is compared to the level measured in a control culture medium lacking the candidate molecule. A compound which promotes an increase in thionein expression (e.g., MT-3) is considered useful in the invention; such a molecule can be used, for example, as a therapeutic for a disease associated with oxidative stress (e.g., those described herein).

If desired, the effect of candidate compounds may, in the alternative, be measured against the level of polypeptide production using the same general approach and standard immunological techniques, such as western blotting or immunoprecipitation with an antibody specific for metallothionein or thionein. For example, immunoassays can be used to detect or monitor the expression of metallothionein or thionein. Polyclonal or monoclonal antibodies which are capable of binding to such a polypeptide can be used in any standard immunoassay format (e.g., ELISA, western blot, or RIA assay) to measure the level of metallothionein. A compound which promotes an increase in the expression of metallothionein or thionein is considered particularly useful. Again, such a molecule can be used, for example, as a therapeutic for a disease associated with oxidative stress.

### Test Compounds and Extracts

In general, compounds capable of treating a disease associated with oxidative stress are identified from large libraries of both natural product or synthetic (or semi-synthetic) extracts or chemical libraries according to methods known in the art. Those skilled in the field of drug discovery and development will understand that the precise source of test extracts or compounds is not critical to the screening methods of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based (e.g., siRNA or microRNA) compounds. Additional compounds that may be used in the screening methods of the invention include any compounds described herein (e.g., chelating agents, modified thionein as well as selenium compounds (e.g., selenocystamine, benzeneselenenyl chloride, and benzeneseleninic acid). Any of these compounds may be chemically modified using methods standard in the art.

Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating diseases associated with oxidative stress should be employed whenever possible.

When a crude extract is found to have a desired activity such as decreasing binding of metal to metallothionein or thionein or increasing expression of thionein, further fractionation of the positive lead extract is necessary to isolate chemical constituents responsible for the observed effect. Thus, the goal of the extraction, fractionation, and purification process is the characterization and identification of a chemical entity within the crude extract having activity that can be useful in treating a disease associated with oxidative stress. Methods of fractionation and purification of such heterogeneous extracts are known in the art. If desired, compounds shown to be useful agents for the treatment of a disease associated with oxidative stress are chemically modified according to methods known in the art.

Modified Thionein with Decreased Metal Binding

The invention also features methods for generating modified thionein (T) with reduced metal (e.g., zinc) binding. In certain embodiments, a modified thionein may further retain the ability to participate in redox reactions as compared to wild-type thionein. Such modified thionein molecules may be useful in the treatment of disease associated with oxidative stress. Modifications may be carried out by any means known in the art. Methods for introducing sequence alterations (e.g., point mutations, insertions, deletions, or any combination thereof) are well known to those skilled in the art.

### Modifications to Thionein

Thionein may be modified at any residue (e.g., by chemical derivitization or point mutation) and may be modified by insertion or deletion of one or more (e.g., 2, 3, 4, 5, 6, 7, 8, 10, 15, or 20) amino acids. Any modification to thionein described herein may be used in the methods of the invention. These modifications may be carried out, for example, by using standard molecular biological techniques. In certain embodiments, lysine residues are altered to generate decreased metal (e.g., zinc) binding. Such alterations may include addition or substitution of a thiol, sulfenic acid, sulfinic acid, sulfonic acid, sulfonate ester, sulfoxide, or sulfone moiety. These lysine residues include the eight lysine residues at positions 20, 22, 25, 30, 31, 43, 51, and 56 of MT1 or MT2 (SEQ ID NOS:1 and 2), lysine residues at positions 21, 26, 31, 32, 44, 47, 52, and 63 of MT3 (SEQ ID NO:3), or lysine residues at positions 21, 28, 32, 44, 52, or 57 of MT4 (SEQ ID NO:4) as these residues are implicated in ATP binding (see Jiang et al., supra) in an as of yet unknown order. As noted above, ATP binding decreases affinity of MT for metal. Thus, modification of the lysine residues in thionein (e.g., those noted above) or residues adjacent to lysine residues, or residues, based on the three dimensional structure of thionein, are in proximity to the lysine residues may be modified and tested for enhanced AMP, ADP, ATP, GSH, or GSSG binding, or any combination thereof. Modifications that increase ATP binding, or binding of other triphosphate nucleotides or analogs thereof, can decrease the affinity of thionein for metal (e.g., zinc) and may therefore be especially useful in the methods of the invention.

Additional exemplary modifications can include substitution of any sulfur atom for selenium. For example, any of the twenty cysteine residues in MT/T may be substituted for either methionine or selenocysteine. Specifically, residues 5, 7, 13, 15, 19, 21, 24, 26, 29, 33, 34, 36, 37, 41, 44, 48, 50, 57, 59, or 60 in MT1 or MT2, residues 6, 8, 14, 16, 20, 22, 25, 27, 30, 34, 35, 37, 38, 42, 45, 49, 51, 64, 66, or 67 in MT3, or residues 6, 8, 14, 16, 20, 22, 25, 27, 30, 34, 35, 37, 38, 42, 45, 49, 51, 58, 60, or 61 in MT4 may be modified. Such modifications can reduce the affinity of thionein for metal, but can, in certain embodiments, allow the modified thionein protein to participate in redox reactions.

In certain embodiments, all cysteine residues are substituted with selenocysteine. Such modified MT/T proteins can be made using solid-phase peptide synthesis or using any technique known in the art or as described below. Also, such methods may employ any MT/T variant (e.g., those described herein).

### Assaying for Metal Binding

Assays for metal affinity and binding can be performed as described herein or as known in the art. Such assays can be used to determine which thionein variants exhibit reduced binding of metals such as zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, or lanthanum as compared to unmodified thionein.

### Assay for Redox Activity

In certain embodiments, a modified thionein is further assayed for redox activity. The precise method employed is not critical to the invention; such measurements can be performed using any method known in the art. The redox state of the N-terminal methionine may be determined, for example, by methods described above including redox potential measurements using modified GFPs or commercially available kits, e.g., as described herein. In other embodiments, redox potential of a protein in solution may be measured using electrodes, e.g., available commercially from Broadley James Corporation, Irvine, Calif.

### Polypeptide Production

Modified thionein polypeptides can be produced by transformation of a suitable host cell with all or part of a thionein-encoding polynucleotide molecule or fragment thereof in a suitable expression vehicle. Those skilled in the field of molecular biology will understand that any of a wide variety of expression systems may be used to provide the thionein polypeptide. The precise host cell used is not critical to the invention. Modified thionein can be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (Pouwels, P. H. et al., 1985, Supp. 1987).

Thionein and thionein fragments, especially those containing amino acids such as selenocysteine, can also be produced by chemical synthesis (e.g., by the methods described in Solid Phase Peptide Synthesis, 2nd ed., 1984 The Pierce Chemical Co., Rockford, Ill.).

Modified thioneins with particular properties (e.g., enhanced ATP binding or reduced metal (e.g., zinc) affinity) may further include chemical modifications such as derivitization of side chain groups, as is known in the art.

Treatment of a Disease Associated with Oxidative Stress

The invention features methods for treating a subject with a disease associated with oxidative stress. The compounds used in the treatment of methods of the invention may, for example, be compounds identified using a screening method described herein, a modified thionein (e.g., as described herein), or a chelating agent.

Diseases Associated with Oxidative Stress

Diseases associated with oxidative stress include Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis.

### Modified Thionein

A modified thionein protein (e.g., identified by the methods of the invention or described herein) may be administered to a subject for treatment a disease associated with oxidative stress. Providing modified thionein to a subject can, by reducing the effects of oxidative stress, treat such a disease associated with oxidative stress.

### Gene Therapy

In addition to administration of a modified thionein protein, expression of polynucleotide encoding thionein (e.g., a modified thionein described herein) can also be induced by introduction of a gene vector into a subject to treat a disease associated with oxidative stress. Any standard gene therapy vector and methodology can be employed for such administration.

### Metal Binding/Chelating Agents

Chelating agents capable of removing metal such as zinc from MT may also be used in the treatment of diseases associated with oxidative stress such as Creutzfeldt-Jakob disease, respiratory distress syndrome, dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis. Such agents will remove metal from MT, thereby allowing the apoprotein T to participate in redox reactions and relieving oxidative stress.

Any chelating agent, including EDTA, EGTA, 1,10-phenanthroline, N,N,N′,N′-Tetrakis(2-pyridylmethyl)ethylenediamine (TPEN), diethyldithiocarbamate (DEDTC), 1,10-phenanthroline, 8-hydroxyquinoline, 8-hydroxyquinoline sulfonate, sodium diethyldithiocarbamate, and 2,2′-bipyridyl may be used in the treatment methods of the invention. Additional chelating agents (e.g., agents that bind zinc or copper), including those of the Zinpyr family, are described, for example, in Goldsmith and Lippard, Inorg. Chem. 45:555-561, 2006; Woodroofe et al., Inorg. Chem. 44:3112-3120, 2005; Woodroofe and Lippard, J. Am. Chem. Soc. 125:11458-11459, 2003; Burdette et al., J. Am. Chem. Soc. 125:1778-1787, 2003; Boerzel et al., Inorg. Chem. 42:1604-1615, 2003; Nolan and Lippard, Inorg. Chem. 43:8310-8317; 2004; Nolan et al., Inorg. Chem. 43:2624-2635, 2004; and Kuzelka et al., Inorg. Chem. 43:1751-1761, 2004. Bis(thiosemicarbazone) agents (e.g., diacetylbis(4-pyrrolidinyl-3-thiosemicarbazone)), which form complexes with zinc, may also be used in the methods of the invention. These reagents are described in greater detail, for example, by Cowley et al. (Chem. Commun. (Camb). 2005(7):845-847, 2005)

### Formulation of Pharmaceutical Compositions

The administration of any compound described herein or identified using the screening methods of the invention can be by any suitable means that results in a concentration of the compound to treat a disease associated with oxidative stress. The compound can be contained in any appropriate amount in any suitable carrier substance, and is generally present in an amount of 1-95% by weight of the total weight of the composition. The composition can be provided in a dosage form that is suitable for the oral, parenteral (e.g., intravenously, intramuscularly, intracranially, intrathecally), rectal, cutaneous, nasal, vaginal, inhalant, skin (patch), ocular, or intracranial administration route. The pharmaceutical compositions can be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions can be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

### Parenteral Compositions

The composition containing compounds described herein or identified using the methods of the invention can be administered parenterally by injection, infusion, or implantation (subcutaneous, intravenous, intramuscular, intraperitoneal, intracranial, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Parenteral compositions used in the methods of the invention can be in a form suitable for sterile injection. To prepare such a composition, the suitable active agent(s) are dissolved or suspended in a parenterally acceptable liquid vehicle. Among acceptable vehicles and solvents that can be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation can also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent can include 10-60% w/w of propylene glycol or the like.

### Nervous System Administration

In many cases, it is desirable that the compound administered be limited to the tissue or tissues affected by the particular disease from which the subject is suffering. In the case of diseases that affect the nervous system, such as Alzheimer's or Parkinson's disease delivery to the affected areas of the nervous system can be achieved, for example, by the methods outlined below.

Treatment of neurodegenerative disease can be hampered by the inability of an active, therapeutic compound to cross the blood-brain barrier (BBB). Strategies to delivery of compounds of the invention (e.g., modified thionein proteins) in such disorders and diseases include strategies to bypass the BBB (e.g., intracranial administration via craniotomy and intrathecal administration), and strategies to cross the BBB (e.g., the use of compounds that increase permeability of the BBB in conjunction with systemic administration of therapeutic compositions, and modification of compounds to increase their permeability or transport across the blood-brain barrier.

Craniotomy, a procedure known in the art, can be used with for delivery of therapeutic compositions to the brain. In this approach, a opening is made in the subject's cranium, and a compound is delivered via a catheter. This approach can be used to target a compound to a specific area of the brain (e.g., the substantia nigra for treating Parkinson's disease or the cortex for treating Alzheimer's disease).

Intrathecal administration provides another means of bypassing the blood brain barrier for drug delivery. Briefly, drugs are administered to the spinal cord, for example, via lumbar puncture or through the use of devices such as pumps. Lumbar puncture is preferable for single or infrequent administration, whereas constant and/or chronic administration can be achieved using any commercially available pump attached to a intraspinal catheter, for example a pump and catheter made by Medtronic (Minneapolis, Minn.).

To allow for delivery across the BBB, compositions of the invention can be administered along with a compound or compounds that induce a transient increase in permeability of the blood-brain barrier. Such compounds include mannitol, Cereport (RMP-7), and KB-R7943, a Na⁺/C^{a++} exchange blocker.

In another embodiments, compounds (e.g., compounds identified using screening methods of the invention) can be modified (e.g., lipidated, acetylated) to increase transport across the blood-brain barrier following systemic administration (e.g., parenteral), by using chemical modifications standard in the art. In one embodiment, compounds of the invention are conjugated to peptide vectors that are transported across the BBB. For example, compounds can be conjugated to a monoclonal antibody to the human insulin receptor as described by Partridge (Jpn. J. Pharmacol. 87:97-103, 2001), thus permitting the compound to be transported across the BBB following systemic administration. Compounds (e.g., those identified using screen methods described herein) can be conjugated to such peptide vectors, for example, using biotin-streptavidin technology. In the case of treatments using a gene therapy vector, in place of or in addition to localizing delivery of the vector, promoters that restrict expression to particular subpopulations of neurons can be employed. For example, expression of a gene therapy vector in treatment of PD can be limited to dopaminergic neurons through the use of a tyrosine hydroxylase promoter.

### Dosages

The dosage of any compound described herein or identified using the methods described herein depends on several factors, including: the administration method, the disease to be treated, the severity of the disorder or disease, whether the disorder or disease is to be treated or prevented, and the age, weight, and health of the subject to be treated.

With respect to the treatment methods of the invention, it is not intended that the administration of a compound to a subject be limited to a particular mode of administration, dosage, or frequency of dosing; the invention contemplates all modes of administration, including intracranial, intrathecal, intramuscular, intravenous, intraperitoneal, intravesicular, intraarticular, subcutaneous, or any other route sufficient to provide a dose adequate to treat the disease associated with oxidative stress. The compound can be administered to the subject in a single dose or in multiple doses. For example, a compound described herein or identified using screening methods of the invention can be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide a sufficient treatment. Conversely, the dosage of the compound can be decreased if the disease is reduced or eliminated.

While the attending physician ultimately will decide the appropriate amount and dosage regimen, a therapeutically effective amount of a compound described herein (e.g., a modified thionein with reduced zinc binding) or identified using the screening methods of the invention, can be, for example, in the range of 0.0035 μg to 20 μg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount can be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound can be, for example, in the range of 100 μg/m² to 100,000 μg/m² administered every other day, once weekly, or every other week. In a desirable embodiment, the therapeutically effective amount is in the range of 1000 μg/m² to 20,000 μg/m², for example, at least 1000, 1500, 4000, or 14,000 μg/m² of the compound administered daily, every other day, twice weekly, weekly, or every other week.

The following example is intended to illustrate rather than to limit the invention.

### EXAMPLE

### Synthesis of Modified Metallothionein or Thionein

In one example, the selenocysteine-substituted metallothionein is synthesized essentially as described in WO00/50448. Briefly, this method includes the following steps: (a) synthesizing the MT or T using a solid support and at least two alpha amino acids having alpha amino groups selected from the group consisting of amino acids with aliphatic group-containing side chains (e.g., hydrogen or alkyl), amino acids with aromatic group containing side chains, amino acids with sulfur group-containing side chains (e.g., a thiol or a thioether), amino acids with hydroxyl group-containing side chains, amino acids with amine group-containing side chains, amino acids with guanidinium group-containing side chains, amino acids with carboxylate-group containing side chains, and amino acids with amide group-containing side chains. The alpha amino groups are protected with Fmoc, t-Boc, or CBZ. The carboxylate groups are protected with a t-butyl ester or a benzyl ester. The hydroxyl groups are protected with a t-butyl ether or a dimethylphosphate ester. The amine groups are protected with a t-Boc or CBZ. The thiol groups are protected with an acetimidomethyl group. Following step (a), step (b) of the method includes cleaving the peptide synthesized in step (a) from the solid support and removing the non-acetimidomethyl protecting groups. Step (c) includes purifying the peptide obtained from step (b), and step (d) includes precipitating the peptide obtained from step (c). Step (e) includes removing the acetimidomethyl protecting group with a solution comprising a silver(I) salt. The primary amino acid sequence of MT or T can differ from wild-type sequence. For example, the amino acid sequence may contain substitution of one or more non-cysteine residues with any naturally occurring or non-naturally occurring amino acid. In certain embodiments, one or more cysteine residues are substituted with selenocysteine. Other modifications include addition of one or more repetitions of the primary sequence of the alpha or beta domain of MT. These repetitions may be fused together in order or any arrangement of alpha and beta domains. The domains may be separated by a spacer sequence of one or more amino acids. In a repeated domain, one or more cysteine residues may be substituted with selenocysteine.

The method step (a) may be accomplished using an automated solid-phase synthesizer. The alpha amino groups may be protected with an Fmoc protecting group, the carboxylate groups may be protected with a t-butyl ester protecting group, the hydroxyl groups may be protected with a t-butyl ether protecting group, and the amine groups may be protected with a t-Boc protecting group.

The cleaving step (b) may be accomplished using a solution comprising about 75 parts by weight phenol, about 28 parts by weight ethanedithiol, about 53 parts by weight thioanisole, about 50 parts by weight water, and about 142 parts by weight trifluoroacetic acid; and the purifying step (c) is accomplished by gel filtration chromatography using a gel prepared from beads comprising dextran that has been cross linked with epichlorohydrin under alkaline conditions where the dry beads have a diameter in a range from about 20 micrometers to about 150 micrometers, and where the gel is prepared and eluted with an aqueous solution comprising 0.1% trifluoroacetic acid. The removing step (e) may be accomplished with a solution comprising silver (I) nitrate in acetic acid.

The produced metallothionein or thionein may be metal containing or metal-free. Where metal containing, the metal may be selected from the group consisting of main group metals, transition metals, lanthanides, and actinides. The metal may also be zinc, copper, gold, cadmium, iron, cobalt, calcium, selenium, manganese, nickel, silver, arsenic, molybdenum, tungsten, aluminum, barium, strontium, bismuth, hafnium, technetium, lanthanum, or a combination thereof.

All patents, patent applications including U.S. Patent Application Nos. 60/787,400, filed Mar. 30, 2006, and 60/839,582, filed Aug. 23, 2006, and publications mentioned in this specification are herein incorporated by reference to the same extent as if each independent patent, patent application, or publication was specifically and individually indicated to be incorporated by reference.

What is claimed is:

1. A polypeptide comprising an amino acid sequence substantially identical to metallothionein or thionein, wherein at least one sulfur atom is substituted with a selenium.

2. The polypeptide of claim 1, wherein said sulfur atom is in a cysteine residue of said polypeptide.

3. The polypeptide of claim 2, wherein ten cysteine residues of said polypeptide are substituted with selenocysteine.

4. The polypeptide of claim 2, wherein all cysteine residues of said polypeptide are substituted with selenocysteine.

5. A polypeptide comprising a fragment of metallothionein or thionein, wherein at least one sulfur atom is substituted with selenium and said fragment is capable of binding a metal.

6. The polypeptide of claim 5, wherein said sulfur is in a cysteine residue of said polypeptide.

7. The polypeptide of claim 5, wherein all cysteine residues of said polypeptide are substituted with selenocysteine.

8. The polypeptide of claim 5, wherein said fragment comprises an α-domain or a β-domain of metallothionein or thionein.

9. The polypeptide of claim 5, wherein said metal is zinc.

10. A method for identifying a candidate compound for treatment of a disease associated with oxidative stress, said method comprising the steps:
(a) contacting a compound with metallothionein and a second polypeptide comprising an amino acid capable of being oxidized; and
(b) measuring the amount of metal released from said metallothionein and the formation of an oxidized amino acid on said second polypeptide in the presence of said compound, wherein a compound that (i) increases the release of metal from metallothionein and (ii) does not substantially increase the amount of said oxidized amino acid in said second polypeptide as compared to in the absence of said compound indicates that said compound is a candidate compound for treatment of a disease associated with oxidative stress.

11. The method of claim 10, wherein said compound is selected from a chemical library.

12. The method of claim 10, wherein said oxidized amino acid is methionine sulfoxide.

13. The method of claim 10, wherein said metal is zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, or lanthanum.

14. The method of claim 13, wherein said metal is zinc.

15. The method of claim 10, wherein said second polypeptide is metallothionein or thionein.

16. The method of claim 15, wherein said oxidized amino acid is methionine sulfoxide.

17. The method of claim 10, wherein said disease is selected from the group consisting of Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis.

18. A method for identifying a candidate compound for treatment of a disease associated with oxidative stress, said method comprising the steps:
(a) contacting a cell or cell extract with a compound; and
(b) measuring the amount metallothionein or thionein in and oxidation state of said cell or cell extract wherein a compound that (i) increases the amount of thionein or decreases the amount of metallothionein and (ii) does not substantially increase the oxidation state of said cell or cell extract as compared to a cell or cell extract not contacted with said compound indicates that said compound is a candidate compound for the treatment of a disease associated with oxidative stress.

19. The method of claim 18, wherein said compound is selected from a chemical library.

20. The method of claim 18, wherein said measuring the oxidation state comprises detecting the presence of an oxidized amino acid.

21. The method of claim 20, wherein said oxidized amino acid is methionine sulfoxide.

22. The method of claim 18, wherein said disease is selected from the group consisting of Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis.

23. A method for identifying a candidate compound for treatment of a disease associated with oxidative stress, said method comprising the steps:
(a) contacting a compound with a cell or cell extract comprising a polynucleotide encoding thionein; and
(b) measuring expression of thionein in said cell or cell extract, wherein an increase in expression in the presence as compared to in the absence of said compound indicates that said compound is a candidate compound for the treatment of a disease associated with oxidative stress.

24. The method of claim 23, wherein said compound is selected from a chemical library.

25. The method of claim 23, wherein said disease is selected from the group consisting of Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis.

26. A method for identifying a thionein variant with a reduced affinity for a metal, said method comprising the steps:
(a) introducing a point mutation, insertion, or deletion into thionein or chemically altering thionein, thereby creating a modified thionein; and
(b) determining the affinity of said metal to said modified thionein, wherein a decreased affinity for said metal indicates that said modified thionein is a thionein variant with reduced affinity for a metal.

27. The method of claim 26, wherein said determining step (b) further comprises measuring the reducing activity of said modified thionein, wherein no substantial decrease in the reducing activity of said modified thionein indicates that said modified thionein is a redox-active thionein variant with a reduced affinity for metal.

28. The method of claim 26, wherein said point mutation comprises a cysteine to selenocysteine point mutation.

29. The method of claim 26, wherein said metal is zinc, copper, cadmium, lead, silver, gadolinium, cobalt, calcium, gold, selenium, arsenic, tungsten, aluminum, manganese, iron, chromium, nickel, molybdenum, barium, strontium, bismuth, hafnium, technetium, or lanthanum.

30. The method of claim 29, wherein said metal is zinc.

31. A method for treating disease associated with oxidative stress, said method comprising administering a thionein variant identified using the method of claim 26 to a patient in need thereof.

32. The method of claim 31, wherein said disease is selected from the group consisting of Alzheimer's disease, Parkinson's disease, Creutzfeldt-Jakob disease, amyotrophic lateral sclerosis, respiratory distress syndrome, muscular dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis.

33. A method for treatment of a patient with a disease associated with oxidative stress, said method comprising administering a chelating agent to said patient, wherein said disease is selected from the group consisting of Creutzfeldt-Jakob disease, respiratory distress syndrome, dystrophy, cataractogenesis, rheumatoid arthritis, progeria, Werner's syndrome, atherosclerosis, diabetes, essential hypertension, cystic fibrosis, regional ileitis (Crohn's disease), macular degeneration, stroke, ischemia, and ulcerative colitis.
